(19) United States
(12) Patent Application Publication
(10) Pub. No.: US 2023/0381357 A1
(43) Pub. Date: Nov. 30, 2023

(54) DEVICES AND METHODS FOR TREATING MICROORGANISMS

(71) Applicant: Scott A. Elrod, Lake Jackson, TX (US)

(72) Inventor: Scott A. Elrod, Lake Jackson, TX (US)

(21) Appl. No.: 18/040,609

(22) PCT Filed: Aug. 4, 2020
(86) PCT No.: PCT/US20/44883

§ 371 Date: Feb. 3, 2023

(51) Int. Cl. A61L 2/20; B01L 1/02; A61L 2/24; F24F 8/24

(52) U.S. Cl. CPC A61L 2/202; B01L 1/025; A61L 2/24; F24F 8/24; B01L 2200/0689; B01L 2300/048; A61L 2202/122; A61L 2202/14; A61L 2202/11; A61L 2202/15; A61L 2202/26

(57) ABSTRACT

Embodiments of the present disclosure relate to devices, systems, and methods for treating microorganisms within an enclosure. The enclosure can be operably coupled to an oxidant source such that oxidant discharged by the oxidant source is disposed within an internal volume defined by the enclosure. The oxidant can modify the microorganisms within the internal volume to render the microorganisms harmless or inert. The oxidant source can discharge a first quantity of oxidant over a first duration of time and subsequently discharge a second lesser quantity of oxidant over a second duration of time to limit the concentration of oxidant to which items within the enclosure are exposed. This process can be repeated for a predetermined duration of time until the microorganisms within the enclosure are sufficiently treated and without damaging the enclosure or items disposed within the enclosure.

### BACKGROUND

Viruses, bacteria, and other microorganisms can cause disease or illness in a living host. These microorganisms can be communicated or transferred from one person to another and spread rapidly across countries and continents. As such, microorganisms can be devastating to a particular demographic of the populous. For example, the elderly or persons having autoimmune deficiencies can be particularly susceptible to being rendered fatally ill by a microorganism. Furthermore, microorganisms can also negatively affect the economic viability of persons who are furloughed or laid off due to the economic impact of a pandemic. Accordingly, reducing the spread of microorganisms, such as viruses, bacteria, and other micro-organisms that cause disease, is desirable.

### SUMMARY

Embodiments of the present disclosure relate to devices, systems, and methods for treating objects to reduce or eliminate microorganisms or scent sources thereon.

An apparatus for treating objects according to at least some embodiments is disclosed. The apparatus includes an enclosure that defines and internal volume. The apparatus includes an oxidant source operably coupled to a wall of the enclosure and configured to output an oxidant within the internal volume. The device includes a controller operably coupled to the oxidant source, the controller being configured to cause the oxidant source to cyclically discharge the oxidant within the internal volume.

An enclosure for treating objects according to at least some embodiments is disclosed. The enclosure includes a set of walls defining an internal volume. The enclosure includes an oxidant source operably coupled to a wall of the set of walls and configured to output an oxidant within the internal volume. The enclosure can include a controller operably coupled to the oxidant source.

The controller can include a computer-readable medium having executable instructions. The controller can include a processor coupled to the computer-readable medium and configured to execute the executable instructions. The executable instructions can cause the controller to perform the operation of outputting, using the oxidant source, a first quantity of oxidant into the internal volume over a first duration of time, the first quantity of oxidant source over the first duration of time correlating to a first phase. The executable instructions can cause the controller to perform the operation of outputting, using the oxidant source, a second quantity of oxidant into the internal volume over a second duration of time, the second quantity of oxidant source over the second duration of time correlating to a second phase. The executable instructions can cause the controller to perform the operation of repeating the first phase and the second phase for a predetermined duration of time to treat microorganisms within the internal volume.

A method of treating objects within an enclosure, according to at least some embodiments, is disclosed. The method includes placing one or more objects in an enclosure, the enclosure having at least one wall defining an interior volume and an oxidant source in fluid communication with the internal volume. The method includes outputting, using the oxidant source, a first quantity of oxidant into the internal volume over a first duration of time, the first quantity of oxidant source over the first duration of time correlating to a first phase. The method includes outputting, using the oxidant source, a second quantity of oxidant into the internal volume over a second duration of time, the second quantity of oxidant source over the second duration of time correlating to a second phase. The method includes repeating the first phase and the second phase for a predetermined duration of time.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1A is a perspective view of an enclosure for treating microorganisms, according to at least some embodiments.

FIG. 1B is a schematic of an oxidant source, according to at least some embodiments.

FIG. 2A is a perspective view of a portable oxidant source, according to at least some embodiments.

FIG. 2B is a front view of the portable oxidant source of FIG. 2A.

FIG. 2C is a bottom view of the portable oxidant source of FIG. 2A.

FIG. 3A is a perspective view of an enclosure for treating microorganisms, according to some embodiments.

FIG. 3B is a front view of the enclosure of FIG. 3A.

FIG. 3C is a sectional view of the enclosure of FIG. 3A.

FIG. 4 is a prospective view of an enclosure for treating microorganisms, according to at least some embodiments.

FIG. 5 is a prospective view of another enclosure for treating microorganisms, according to at least some embodiments.

FIG. 6 is a prospective view of yet another enclosure for treating microorganisms, according to at least some embodiments.

FIG. 7 is a flow diagram of a method of treating microorganisms within an enclosure, according to at least some embodiments.

### DETAILED DESCRIPTION

Embodiments of the present disclosure relate to devices, systems, and methods for treating objects within an enclosure. For example, an internal volume within the enclosure can be treated to render microorganisms within the enclosure subsequently harmless, inert, or otherwise uncommunicable to persons and/or animals. Additionally, any scent sources or scent molecules on the objects may be rendered inert or otherwise remove scent therefrom. Any type of enclosure is contemplated herein, for example, the enclosures described herein can be boxes, lockers, closets, operating rooms, vehicles, tents, enclosed shelving, luggage, bags, containers, or any other types of enclosures now known in the art or subsequently developed.

The enclosure can be operably coupled to an oxidant source such that oxidant discharged by the oxidant source is disposed within the internal volume of the enclosure. The oxidant can modify the microorganisms within the internal volume to render the microorganisms harmless or inert. For example, in some embodiments, influenza, COVID-19, another virus, or a combination thereof can be present in the enclosure and the oxidant can include ozone which reacts with the viruses to render them harmless or inert. More specifically, the oxidant (e.g., ozone) can transfer an oxygen atom to the microorganism to change the microorganism's molecular composition. Additionally, oxidant can react with scent sources (e.g., bacteria and fungi) and scent molecules to render them incapable of producing more scent or altering the scent to reduce or eliminate the scent therefrom.

As such, the term “treat” can refer to one or more of sanitizing or cleaning an enclosure and objects within the enclosure to render microorganisms within the enclosure harmless or inert and any reduce or eliminate scent sources or scent molecules within the enclosure.

More specifically, items disposed within the internal volume of the enclosure can be treated as to alter or otherwise modify microorganisms on or around the items to render the microorganisms harmless or inert or to at least partially reduce (e.g., eliminate) any scent sources or scent molecules thereon. In other words, the items can be treated to mitigate or eliminate the harmful effects of microorganisms disposed on the items and thereby allow the items to be safely worn or used by one or more persons. For example, the enclosure can be a storage cabinet at a hospital in which medical equipment (e.g., scrubs, face-masks, face-shields, lab coats, etc.) can be disposed to be treated to render microorganisms like COVID-19 inert or harmless to the medical professionals who use the medical equipment. Additionally, items can be treated to reduce or eliminate scent sources or scent molecules thereon to allow the items to be used without spreading scent.

In some examples, the enclosure can be a hospital room that has recently been vacated by a patient. While the hospital room can be cleaned after the patient vacates, fluids, bacteria, mold, fungi, viruses, and other contaminants can remain in the ambient air, mattress, drapes, and other objects within the hospital room. However, an oxidant source can be placed within the hospital room and operated to discharge oxidant to render microorganisms in the hospital room inert or otherwise harmless to medical professionals and ensuing patients. A surgical suite is another example of an enclosure that may need to be treated to eliminate or substantially reduce harmful microorganisms.

The oxidant source can be a portable unit which can be disposed within the enclosure and/or removably coupled to the enclosure. In an embodiment, the oxidant source can be removably coupled to a wall or ceiling of the enclosure. In embodiments, the oxidant source can be defined by multiple distinct oxidant sources, for example, the oxidant source can include distinct oxidant sources positioned throughout a heating, ventilation, and air conditioning (HVAC) system of a hospital or stadium. The size and portability of the oxidant source can relative to the volume of the enclosure that needs to be treated for microorganisms. Thus, an operating suite may require an oxidant source integrated into the air handling system while a gym locker or gym bag may require a relatively small, lightweight, and portable oxidant source.

Some materials, like certain types of polymers, can be damaged during prolonged exposure to high concentrations of an oxidant such as ozone. For example, exposure to high concentrations of an oxidant can cause some transparent polymers to occlude or otherwise turn opaque. One aspect of the present disclosure relates to cyclically discharging oxidant into the internal volume of the enclosure to limit or prevent degradation of the items within the enclosure due to the oxidant. For example, a face-shield can be treated within the disclosure without damaging or removing a layer of anti-fog compound disposed on a surface of the face shield.

In some embodiments, a first quantity of oxidant can be discharged over a first duration of time. Thereafter, a second lesser quantity of oxidant can be discharged over a second duration of time to limit the concentration of oxidant to which items within the enclosure are exposed. This process can be repeated for a predetermined duration of time until the microorganisms within the enclosure are sufficiently treated and without damaging items within the enclosure.

Accordingly, the particular items within the enclosure can dictate the first and second quantities of oxidant as well as the first and second durations of time. For example, if the items within the enclosure, or the enclosure itself, are particularly susceptible to being damaged by the oxidant(s), the first and second quantities of oxidant can be reduced while the first and second durations of time can be increased to avoid exposing the items to a concentration of oxidant that could damage the items. If, however, the items within the enclosure, or the enclosure itself, are resilient to the effects of the oxidant, the first and second quantities of oxidant can be increased and the first and second durations of time can be reduced.

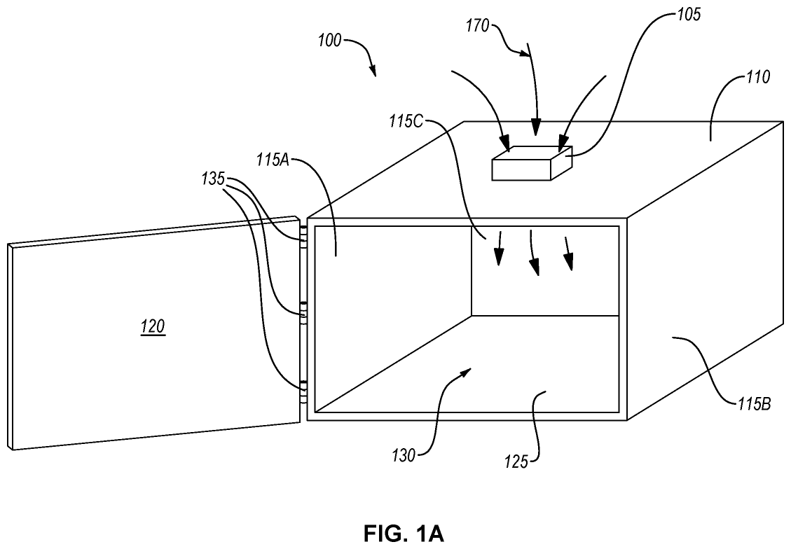

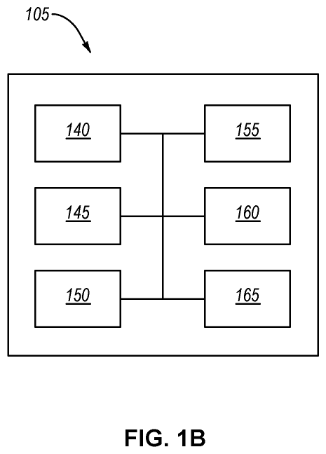

FIG. 1A shows an example of an enclosure 100 for treating microorganisms, according to at least some embodiments. The enclosure 100 includes an oxidant source 105 positioned on a ceiling 110 or top of the enclosure 100. The enclosure 100 can include one or more sidewalls 115A, 115B, 115C, a door 120, and a floor 125 or bottom of the enclosure 100. The ceiling 110, the one or more sidewalls 115A, 115B, 115C, the door 120, and the floor 125 can form an internal volume 130 within the enclosure 100. The internal volume 130 can be in fluid communication with the oxidant source 105 such that the oxidant source 105 can discharge oxidant into the internal volume 130.

The door 120 can be pivotably coupled to the enclosure 100, for example, by one or more hinges 135 pivotally coupling the door 120 to one or more of the sidewalls 115A, 115B, 115C. The door 120 can permit access to the internal volume 130 of the enclosure 100 such that objects (e.g., medical equipment/hunting equipment) and/or persons can be disposed within the internal volume 130. In some embodiments, the enclosure 100 can include multiple doors, such as a set of double doors pivotally coupled to opposing sides of the enclosure.

The oxidant source 105 can be positioned over a through-hole (not shown) formed within the ceiling 110 such that oxidant discharged from the oxidant source 105 is disposed within the internal volume 130 of the enclosure 100. Although the oxidant source 105 is illustrated as being positioned on the ceiling 110 in FIG. 1A, the oxidant source 105 can alternatively, or additionally, be positioned on one or more of the sidewalls 115A, 115B, 115C, the door 120, and/or the floor 125. In some embodiments, the oxidant source 105 can draw ambient air adjacent the enclosure 100 into the internal volume 130 (as represented by air-flow lines 170). For example, the oxidant source 105 can include one or more fans (not shown) which draw ambient air through the oxidant source 105 and into the internal volume 130.

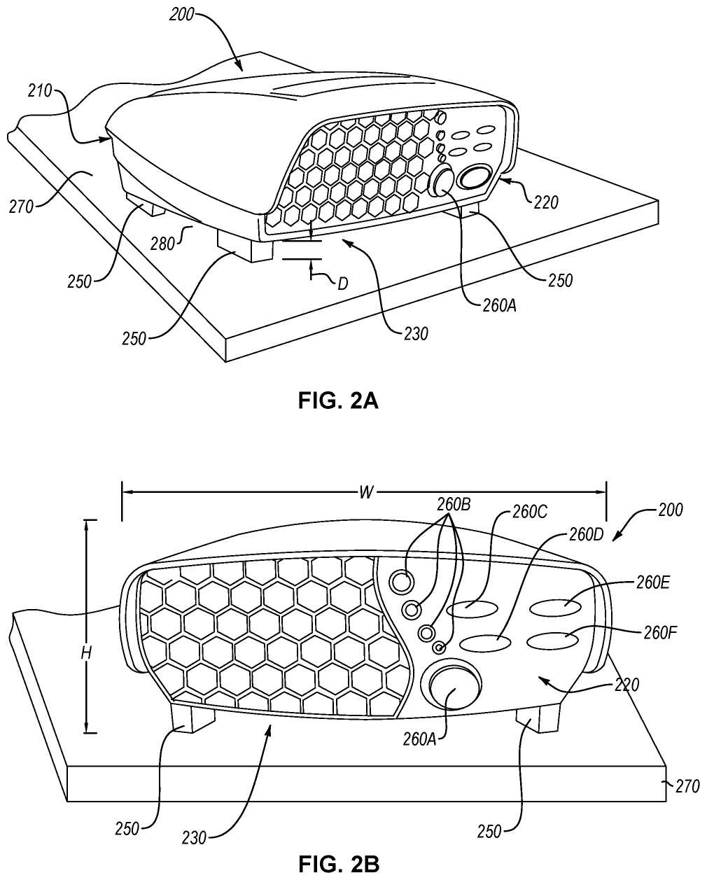

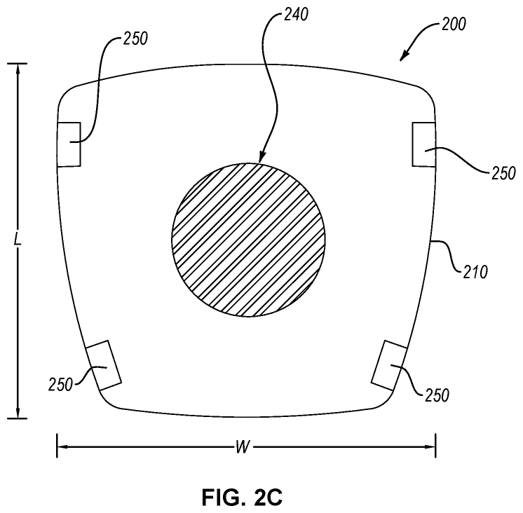

In some embodiments, the oxidant source 105 can be operably coupled to a controller (not shown). For example, the controller can be incorporated into a housing of the oxidant source 105 and include one or more processors and memory storage having one or more operational programs stored therein. When executed by the processor, the one or more operational programs can cause the oxidant source 105 to operate. For example, an operational program can cause the oxidant source 105 to discharge oxidant into the internal volume 130 for a predetermined duration of time. Additionally, or alternatively, the rate at which oxidant is discharged into the internal volume 130 can be cyclically varied over the predetermined duration of time to preserve items disposed within the internal volume 130 while sufficiently treating microorganisms within the internal volume 130. In some embodiments, the oxidant is ozone and the average concentration of ozone within the internal volume 130 is about 0.5 ppm for a predetermined duration of about 30 minutes. Addition detail and disclosure relating to cyclically discharging oxidant into the enclosure 100 will be described herein with reference to FIGS. 2A-5.

FIG. 1B shows a schematic of the oxidant source 105, according to at least some embodiments. The oxidant source 105 can include a controller 140, a user interface module 145, a fan 150, a power supply 155, a communication module 160, and an oxidant generator 165. Each of the components of the oxidant source 105 is discussed in more detail below.

The controller 140 is operably coupled to the oxidant source 105 to control generation or emission of oxidant, according to output parameters in one or more operational programs. As such, the controller 140 can be communicatively coupled to one or more of the user interface module 145, the fan 150, the power supply 155, the communication module 160, or the oxidant generator 165. The controller 140 includes one or more operational programs stored therein to control one or more output parameters of the oxidant source 105, such as amount of oxidant produced or emitted (e.g., per unit time), emission durations, or pulse durations. The one or more operational programs include machine readable and executable instructions to control output of an oxidant from the oxidant generator 165. The machine readable and executable instructions control output of an oxidant from the oxidant source 105, such as via selective control of electrical bias supplied to the oxidant generator 165. Each of the one or more operational programs include oxidant output parameters. For example, the operational programs can cause the power supply 155 to provide a voltage to the oxidant generator 165 and thereby generate oxidant at a rate which correlates with the provided voltage.

The user interface module 145 can receive input from a user of the oxidant source 105 and communicate signals correlating with the input to the controller 140. For example, a user of the oxidant source 105 can input a desired mode of operation (e.g., operational program) via one or more input mechanisms (e.g., buttons, knobs, switches, etc.) of the user interface module 145. Addition detail and disclosure relating to selecting operational programs at a user interface will be described herein with reference to FIGS. 2A-5.

In examples, the fan 150 can be one or more fans, such as an intake fan, a cooling fan, an output fan, etc. Exemplary fans include microfans, centrifugal fans, cyclonic blowers, etc. Each fan may be operably coupled to the power supply 155 and the controller 140, to activate, adjust speed, and deactivate according to operational instructions. The fan 150 can cause ambient air external to the oxidant source 105 to be drawn into the oxidant source 105. In examples, air that is drawn into the oxidant source 105 can be mixed with an oxidant generated by the oxidant generator 165 and propelled from the oxidant source 105 into an enclosure. In examples where the oxidant generator includes a corona discharge ozone generator, the air may be drawn across corona discharge coils by the fan 150. Addition detail and disclosure relating to the functionality of the fan 150 will be described herein with reference to FIGS. 2A-5.

The power supply 155 may be operably coupled to the controller 140, the user interface module 145, the fan 150, the communication module 160, the oxidant generator 165, or any other components of the oxidant source 105. For example, the power supply 155 may include one or more batteries (e.g. lithium-ion, nickel-cadmium, nickel-metal hydride, etc.) or portable chargers (e.g., power banks). The one or more batteries may be rechargeable. In examples, the one or more batteries may be modular battery packs, which may be removed and replaced. In examples, the one or more batteries have a connection for charging, such as a connection for the portable charger. In some examples, the power supply 155 may include a solar cell or a connection for a solar cell.

The power supply 155 may be a replaceable and rechargeable battery, such as a 12 volt battery. The rechargeable battery may be a lithium ion battery, lithium-ion polymer, a nickel-cadmium battery, nickel-metal hydride, lead acid, etc., batteries. The power supply 155 may include a plurality of rechargeable batteries. The rechargeable battery may be at least a 1 volt battery, such as 1.5 volts to 3 volts, 3 volts to 6 volts, 6 volts to 9 volts, 9 volts to 12 volts, 12 volts to 15 volts, 15 volts to 24 volts, greater than 12 volts, less than 24 volts, or less than 15 volts.

In examples, the power supply 155 may include a cord or wired connection for connecting to a power outlet. For example, the power supply 155 may include 110 volt, 220 volt, or similar connections. The cord may allow the user to plug the oxidant source 105 into a power outlet in a room, an extension cord, or a power station or power bank (e.g., battery pack or bank). Accordingly, the power supply 155 may include a wall outlet, the extension cord, or a power station or power bank. In examples, the power supply 155 may include both a wired connection for coupling to a power source and a battery pack. Accordingly, the oxidant source 105 may be run with our without battery power. In examples, the wired connection may be provided as a detachable power cord which may be removed from the oxidant source 105. The wired connection may serve to recharge the battery pack and provide power to the oxidant source 105.

The communication module 160 can be operably configured to send and receive operational instructions relative to the oxidant source 105. For example, the wireless communication module can communicatively couple the oxidant source 105 to one or more electronic devices, such as a cellular phone, a laptop, a tablet, a proprietary remote control configured to specifically communicate with the oxidant source 105, a smart watch, or the like. In some embodiments, the user interface module 145 may be at least partially incorporated into the one or more electronic devices, such as in application software stored thereon. As such, any of the operational programs disclosed herein can be implemented via the one or more electronic devices.

The oxidant generator 165 may include an ozone generator such as corona discharge ozone generator (e.g., corona discharge plate), an ultraviolet ozone generator, an electrolytic ozone generator, or any other type of ozone generator. In some examples, the oxidant source 105 includes an ionizer or electrostatic precipitator. The corona discharge ozone generator presents the advantages of being relatively small and efficient in comparison to other oxidant generators. The oxidant source 105 may include a source of peroxides or derivatives thereof (e.g., hydroperoxides, hydroxyl radicals, or peroxide radicals). For example, a catalytic ionizer may provide oxidants. Catalytic ionization of air by ultraviolet light may produce a mixture of hydroxyl ions, hydroxyl radicals and hydrogen peroxide ions (as well as ozone). The oxidant generator 165 may be an activated water or peroxide ion or radical generator, such as an electrolytic device for carrying out electrolysis of one or more of water or a peroxide. The oxidant source 105 may include a fluid oxidant storage and a mist sprayer operably coupled thereto to spray a mist (e.g., droplets or micro droplets) of fluid oxidant. In examples, suitable portable oxidant sources may include those found in the HR200, HR230, or HR300 ozone generators from Ozonics LLC, of Mason City, Iowa, U.S.A.

FIGS. 2A-2C show multiple views of a portable oxidant source 200. The portable oxidant source 200 can include a housing 210, a user input region 220, an oxidant output port 230, an air-intake port 240, and one or more support structures 250. The portable oxidant source 200 can be substantially similar to the oxidant source 105 described herein with reference to FIG. 1B. For example, the portable oxidant source 200 can include the controller 140 operably coupled to at least one of the user interface module 145, the fan 150, the power supply 155, the communication module 160, or the oxidant generator 165. In some embodiments, the portable oxidant source 200 can be lightweight (e.g., less than 3 lbs.) and easily carried by a person to be used in a variety of enclosures (e.g., bags, containers, cupboards, closets, and so on). Accordingly, the portable oxidant source 200 can have a small form-factor or footprint as to easily be accommodated within an enclosure. For example, in some embodiments, the portable oxidant source 200 can be less than 200 mm in width W, less than 200 mm in length L, and less than 100 mm in height H.

The housing 210 can carry or support one or more of the operational components of the portable oxidant source 200. For example, at least one of the controller 140, the user interface module 145, the fan 150, the power supply 155, the communication module 160, the oxidant generator 165, or another component can be disposed within the housing 210. The housing 210 may be made of a polymer (e.g., high density polyethylene, high density polystyrene, or polycarbonate), a composite (e.g., fiberglass or carbon fiber), a metal (e.g., steel, aluminum, alloys), a ceramic or cermet, any other material capable of withstanding impacts and preventing crushing of the contents of the housing 210, or combinations of any of the foregoing.

In examples, the housing 210 can form one or more apertures defining air intakes (e.g., air-intake port 240), output ports (e.g., oxidant output port 230), or device ports (e.g., hole for user interface, hole for electrical inputs, hole for battery port). For example, the housing 210 may include a hole positioned and sized to accommodate a battery therein. In such examples, the power supply may include a replaceable battery pack and the hole (e.g. port) may accommodate removal and replacement of battery packs. In examples, the one or more apertures define a grill for an air intake (e.g., air-intake port 240) and/or an output port (e.g., oxidant output port 230).

The user input region 220 can be an interface at which a user of the portable oxidant source 200 inputs parameters to the controller 140. As such, the user input region 220 can be coupled to or integrated with the user interface module 145. The user input region 220 may include one or more inputs, such as buttons, switches, knobs, indicators, or toggles, for activating the portable oxidant source 200, deactivating the portable oxidant source 200, selecting a mode of operation, increasing or decreasing an output of the portable oxidant source 200, viewing an attribute of the portable oxidant source 200, or directing any other operation of the portable oxidant source 200. For example, the user input region 220 can include a power button 260A that can be actuated to transition the device 200 between an “on state” wherein the portable oxidant source 200 is operative and an “off state” wherein the portable oxidant source 200 is inoperative.

In some embodiments, the user input region 220 can also include one or more indicators, such as LEDs, that indicate an attribute of the portable oxidant source 200. For example the user input region 220 can include indicators 260B which display the current charged state of the power supply. Each of the indicators 260B can represent a quarter of a total electrical potential of a battery of the power supply. Thus, all four of the indicators 260B will be illuminated when the battery is in a fully charged state, three of the indicators 260B will be illuminated when the battery is at a 75% charged state, and so on.

Additionally, or alternatively, the indicators 260B can express an amount of time (e.g., run-time) the portable oxidant source 200 will continue to emit oxidant. For example, all four of the indicators will be illuminated when more than 75% of the run-time remains, three of the indicators 260B will be illuminated when between 50% and 75% of the run-time remains, and so on.

The user input region 220 can also include one or more buttons, switches, knobs, or toggles, which cause the portable oxidant source 200 to operate based on operational programs stored on the controller 140 (e.g., a memory storage). For example, the user input region 220 can include a first button 260C that causes the portable oxidant source 200 to operate based on a first operational program. The first operational program can cause the portable oxidant source 200 to output a quantity of oxidant over a pre-determined duration of time. For example, the first operational program may cause the portable oxidant source 200 to output 1 gram of oxidant over a two-hour duration of time at a constant output rate (i.e., an oxidant output rate of 500 mg per cubic meter per hour).

As shown in FIGS. 2A and 2B, the user input region 220 can also include a second button 260D that causes the portable oxidant source 200 to operate based on a second operational program stored on the controller 140. The second operational program can cause the portable oxidant source 200 to output a quantity of oxidant over a pre-determined duration of time different from the first operational program. For example, the second operational program may cause the portable oxidant source 200 to output at least 1 g of oxidant over a four-hour duration of time at a constant output rate (i.e., an oxidant output rate of 250 mg per cubic meter per hour). In some embodiments, the quantity of oxidant output associated with the first operational program is different from the quantity of oxidant output associated with the second operational program. For example, the quantity of oxidant output associated with the second operational program can be less than (e.g., about half of) the quantity of oxidant output associated with the first operational program.

In some embodiments, the user input region 220 can include a third button 260E that causes the portable oxidant source 200 to operate based on a third operational program stored on the controller 140. The third operational program can cause the portable oxidant source 200 to cyclically output a plurality of quantities of oxidant over a pre-determined duration of time. Accordingly, the third operational program may cause the portable oxidant source 200 to output oxidant at a relatively high rate for a first duration of time (i.e., a first phase), subsequently output oxidant at a relatively low rate for a second duration of time (i.e., a second phase), and cyclically repeat the first and second phases for a pre-determined duration of time. For example, the portable oxidant source 200 can output at least 60 mg of oxidant per cubic meter per hour over a 3.5 minute duration of time during the first phase, subsequently output about 6 mg (or less) of oxidant per cubic meter per hour over a 1 minute duration of time during the second phase, and then alternately repeat the first and second phases for a 22.5 minute duration of time (i.e., each of the first and second phases are repeated 5 times). By alternating to a phase wherein oxidant is output at a relatively lower rate (e.g., the second phase), an item within the enclosure can be protected from damage caused by constant or near constant exposure to relatively high concentrations of oxidant, such as ozone, while still treating microorganisms or scent sources on the item.

In some embodiments, the user input region 220 can include a fourth button 260F that causes the portable oxidant source 200 to operate based on a fourth operational program stored on the controller 140. Like the third operational program, the fourth operational program can cause the portable oxidant source 200 to cyclically output a plurality of quantities of oxidant over a pre-determined duration of time. Accordingly, the fourth operational program may cause the portable oxidant source 200 to output oxidant at a relatively high rate for a first duration of time (i.e., a first phase), subsequently output oxidant at a relatively low rate for a second duration of time (i.e., a second phase), and cyclically repeat the first and second phases for a pre-determined duration of time. For example, the portable oxidant source 200 can output at least 60 mg of oxidant per cubic meter per hour over a 5.5 minute duration of time during the first phase, subsequently output 4-10 mg of oxidant per cubic meter per hour over a 0.5 minute duration of time during the second phase, and then repeat the first and second phases for a 30 minute duration of time (i.e., each of the first and second phases are repeated 5 times). The output parameters (e.g., quantities of oxidant discharged and the durations of time) of the fourth operational program can be efficient in treating items within an enclosure that are made from materials that are less prone to damage when exposed to an oxidant, such as polyurethane.

By utilizing multiple phases with an initial, higher oxidant output and at least a second, lower oxidant output, in repeating cycles, the oxidant concentration can be reduced to prevent damage to the objects being treated, while maintaining at least a minimum oxidant concentration to prevent reintroduction of contaminants via circulation of air through the treatment device. Accordingly, the devices, systems, and methods herein may be used to treat objects without damaging the objects (e.g., frosting face shields) while also preventing recontamination of the objects. The output parameters may be controlled to allow treatment of various objects (e.g., material types) and contaminants thereon.

In examples, output parameters of the one or more operational programs (e.g., quantities of oxidant discharged and their associated durations of time) stored in the controller 140 are composed to direct a selected amount of oxidant output per unit time. For example, the oxidant emission rate may be at least 1 mg of oxidant (e.g., ozone) per cubic meter per hour, such as 1 mg/m³ per hour to 10 g/m₃ per hour, 10 mg/m₃ per hour to 100 mg/m₃ per hour, 100 mg/m₃ per hour to 1 g/m₃ per hour, 1 g/ m₃ per hour to 2 g/m₃ per hour, 2 g/m₃ per hour to 3 g/m₃ per hour, 3 g/m₃ per hour to 4 g/m₃ per hour, 4 g/m₃ per hour to 5 g/m₃ per hour, 5 g/m₃ per hour to 6 g/m₃ per hour, 6 g/m₃ per hour to 7 g/m₃ per hour, 7 g/m₃ per hour to 8 g/m₃ per hour, 8 g/m₃ per hour to 9 5 g/m₃ per hour, 9 g/m₃ per hour to 10 g/m₃ per hour, 10 g/m₃ per hour to 11 g/m₃ per hour, 11 g/m₃ per hour to 12 g/m₃ per hour, 12 g/m₃ per hour to 15 g/m₃ per hour, less than 15 g/m₃ per hour, less than 10 g/m₃ per hour, or less than 1 g/m₃ per hour. Any combination of the preceding values may be utilized for the first and second phase for any of the durations of time disclosed herein, respectively.

In some embodiments, the output parameters may include a voltage delivered to a corona discharge plate (e.g., electrodes) of the portable oxidant source 200. The output parameter for the voltage delivered to the corona discharge plate may be at least 1 volt, such as 1 volt to 10,000 volts, 100 volts to 3,000 volts, 3,000 volts to 6,000 volts, 6,000 volts to 10,000 volts, less than 6,000 volts, less than 5,000 volts, at least 1,000 volts, at least 3,000 volts, or at least 4,000 volts.

In examples, the output parameters of the one or more operational programs stored in the controller 140 are composed to direct a selected amount of oxidant output per unit time for a selected duration. For example, the output parameters may include an emission duration of one or more pulses of oxidant in one or more phases of at least a 5 second duration, such as 5 seconds to 12 hours, 30 seconds to 6 hours, 1 minute to 3 hours, 5 minutes to 1 hour, 10 seconds to 10 hours, 10 seconds to 10 minutes, 30 seconds to 5 minutes, 1 minute to 3.5 minutes, less than 6 hours, or less than one hour. The pulse durations may be at least 20 seconds, such as 20 seconds to 1 hour, 1 minute to 3 hours, 1 minute to 40 minutes, 2 minutes to 30 minutes, 3 minutes to 20 minutes, 30 seconds to 10 minutes, 5 minutes to 15 minutes, 5 minutes to 20 minutes, 20 minutes to 40 minutes, 40 minutes to an hour, 30 seconds to 5 minutes, 1 minute to 3.5 minutes, less than an hour, less than 30 minutes, or less than 20 minutes. The pulses may be delivered according to a relatively constant amount and duration of oxidant emission during a respective phase.

In embodiments, the one or more buttons of the user input region 220 can initiate respective operational modes of the oxidant source 200. For example, the one or more buttons of the user input region 220 can cause the portable oxidant source 220 to operate in a standard mode, boost mode, and/or hyperboost mode. The hyperboost mode can cause the portable oxidant source 200 to discharge or output oxidant at a relatively higher rate than the boost mode. Similarly, the boost mode can cause the portable oxidant source 200 to discharge or output oxidant at a relatively higher rate than the standard mode. In embodiments, the hyperboost mode may cause the portable oxidant source 200 to output oxidant at a rate sufficient to cause a concentration of oxidant within an enclosure to reach about 0.5 ppm to about 20 ppm within 10 minutes, 30 minutes, 60 minutes, or less than 10 minutes. In embodiments, the boost mode may cause the portable oxidant source 200 to output oxidant at a rate sufficient to cause a concentration of oxidant within an enclosure to reach about 0.5 ppm to about 11 ppm within 10 minutes, 30 minutes, 60 minutes, or less than 10 minutes. In embodiments, the standard mode may cause the portable oxidant source 200 to output oxidant at a rate sufficient to cause a concentration of oxidant within an enclosure to reach about 0.5 ppm to about 8 ppm within 10 minutes, 30 minutes, 60 minutes, or less than 10 minutes. Each of the standard, boost, and hyperboost modes can include cyclically increasing and decreasing a quantity of oxidant output from the portable oxidant source 200 to prevent damage to objects exposed to the oxidant output by the portable oxidant source 200.

The output parameters may include actuation of the fan 150. As such, the output parameters may cause the fan to operate respective to various fan speeds or fan actuation durations. Each fan may be operably coupled to the power supply 155 and the controller 140, to activate, adjust speed, and deactivate according to operational instructions. For example, an output fan may be disposed adjacent to the oxidant output port 230 of the portable oxidant source 200 to propel the oxidant therefrom. Additionally, or alternatively, an intake fan can be positioned in the housing 210 adjacent to the portable oxidant source 200 to draw air therethrough. The intake fan may provide an increase in oxidant output (e.g., ozone) by drawing elemental oxygen through electrodes (e.g., corona discharge plate) of the portable oxidant source 200 when compared to a portable oxidant source without a fan.

In examples, the fan 150 can operate while the portable oxidant source 200 is not generating oxidant. For example, ozone may remain on the corona discharge coils of a corona discharge ozone generator. In such examples, the ozone may degrade the coils if left in place. Ozone degradation may cause the ozone generator to lose efficiency and drain the battery of the portable oxidant source. A short purge with ambient air may help void the coils of any ozone after production of ozone is halted. The fan 150 may remain in operation for at least at least 1 second after the portable oxidant source 200 has ceased producing oxidant, such as 2 seconds to 2 minutes, 3 seconds to 10 seconds, 5 seconds to 15 seconds, 10 seconds to 20 seconds, 15 seconds to 30 seconds, 2 seconds to 30 seconds, 30 seconds to 1 minute, 1.5 minutes, 1.5 minutes to 2 minutes, less than 2 minutes, or less than 1 minute after the portable oxidant source 200 has ceased producing oxidant.

In examples, the fan 150 can remain in operation at the conclusion of each operation program to void residual oxidant from settling or collecting within the portable oxidant source 200. For example, the fan 150 can continue to operate for a duration of time at the conclusion of any one of the first, second, third, or fourth operational programs described herein after the oxidant generator has ceased outputting/creating oxidant. Additionally or alternatively, the fan 150 can continue to operate for a duration of time at the conclusion of any one of the first, second, third, or fourth operational programs to move ambient air through the portable oxidant source 110 such that ambient air is circulated within the enclosure.

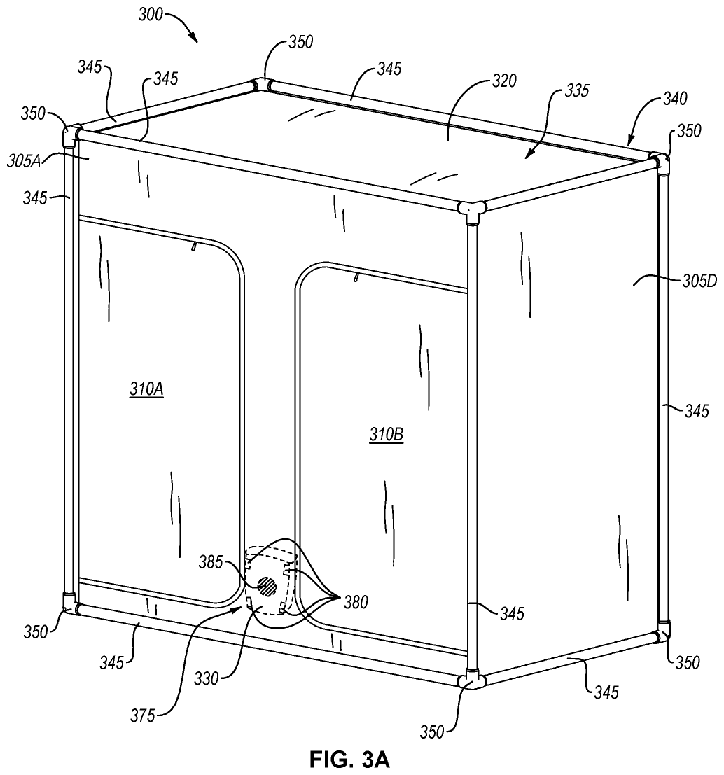

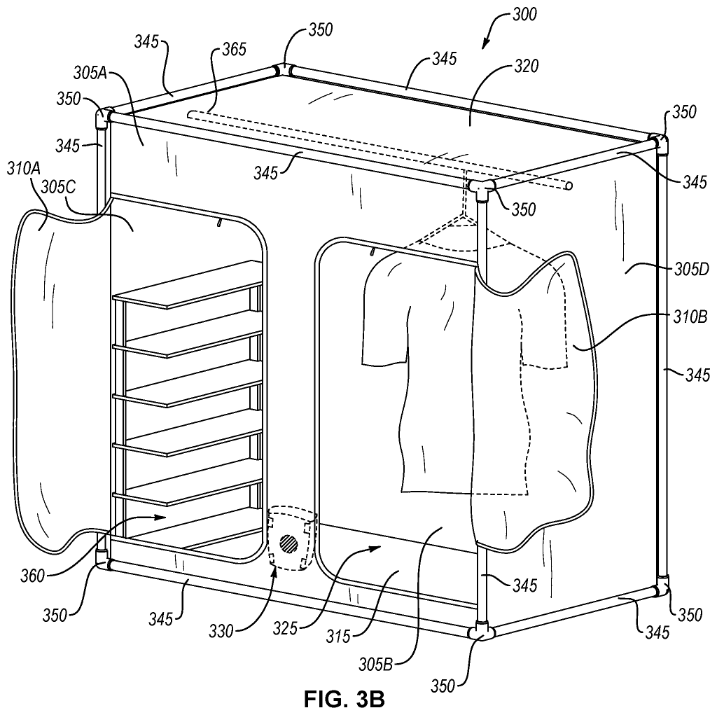

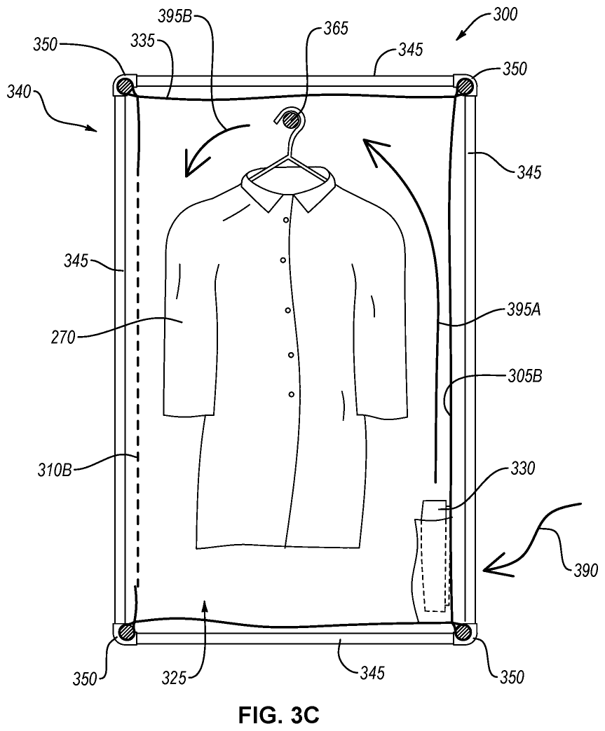

The one or more support structures 250 can extend from the housing 210 of the portable oxidant source 200. In examples, the one or more support structures 250 and at least a portion of the housing 210 can be formed of a singular piece of material, such as, an injection molded polymer. The one or more support members 250 can additionally, or alternatively, be fastened or affixed to the housing 210. In examples, the one or more support structures 250 can extend from the housing 210 to prevent objects from obstructing airflow to the air-intake port 240. For example, the one or more support structures 250 can retain an aperture formed within a fabric enclosure in an open state and thereby prevent the fabric enclosure from obstructing the air-intake port 240 (see FIG. 3B).

The support structures 250 can be configured to support the housing 210 at an elevated position relative to a surface 270 on which the portable oxidant source 200 is positioned. While supported at an elevated position, a gap or space 280 can be formed under the housing 210 such that ambient air can be drawn into the air-intake port 240 formed in the housing 210. Each of the one or more support structures 250 can displace the air-intake port 240 a distance D from the surface 270 to provide an air-flow path to the air-intake port 240. The distance D can relate to a size of the one or more support structures 250. For example, the distance D and size can be equivalent. In some embodiments, the size of each support structure 250 can be dissimilar from one or more of the other support structures 250. For example, the support structures 250 positioned furthest away from the air-output port 230 can be shorter than the support structures 250 positioned nearest the air-output port 230. In this example, the distance D would be equivalent to the average size of the support structures 250. The distance D between the air-intake port 240 and the surface 270 can be at least 5 millimeters (mm), such as 10 mmm to 100 mm, 20 mm to 75 mm, 30 mm to 60 mm, 50 mm, or less than 100 mm.

FIGS. 3A-3C show various views of an enclosure 300 for treating microorganisms, according to some embodiments. The enclosure 300 can include one or more sidewalls 305A, 305B, 305C, 305D one or more doors 310A, 310B, a floor 315 or bottom of the enclosure 300, and a ceiling 320 or top of the enclosure 300. The one or more sidewalls 305A, 305B, 305C, 305D, the floor 315, and the ceiling 320 can form an internal volume 325 within the enclosure 300. The internal volume 325 can be in fluid communication with an oxidant source 330 such that the oxidant source 330 can discharge oxidant into the internal volume 325. The oxidant source 330 can be substantially similar to the oxidant source 105 and the portable oxidant source 200 described herein. For example, the oxidant source 330 can include the controller 140 operably coupled to at least one of the user interface module 145, the fan 150, the power supply 155, the communication module 160, or the oxidant generator 165.

FIGS. 3A-3C illustrate an example embodiment, wherein the sidewalls 305A, 305B, 305C, 305D, the floor 315, and the ceiling 320 of the enclosure 300 include a fabric 335 or other flexible material coupled to a modular frame 340. In this example, the modular frame 340 includes multiple linear components 345 interconnected via three-way connectors 350 at each corner of the enclosure 300. The fabric 335 can include sleeves which receive the linear components 345 of the modular frame 340 to retain the fabric 335 in a fixed position relative to the modular frame 340. This embodiment can provide an enclosure 300 that is light weight and easily disassembled, yet has a relatively large internal volume 325 for housing objects (e.g., equipment such as clothing or personal protective equipment) exposed to microorganisms.

The one or more doors 310A, 310B can provide access to the internal volume 325. For example, the doors 310A, 310B can be unzipped to form openings within the sidewall 305A to provide access to shelves 360, a bar 365, and/or other objects within the internal volume 325. Although the embodiment illustrated in FIGS. 3A-3C depict shelving and a bar for hanging articles of clothing, any mechanism for retaining objects can be disposed within the enclosure 300. For example, shelves, hooks, hangers, drawers, pegs, or any other mechanism for retaining objects within the enclosure 300.

In some embodiments, the enclosure 300 can treat objects 370 (e.g., medical equipment such as masks, goggles, face shields, stethoscopes, lab coats, scrubs, and the like) positioned on the shelves 360 or hung from the bar 365 within the internal volume 325. For example, the oxidant source 330 can be coupled to the enclosure 300 as to discharge oxidant onto the objects (e.g., medical equipment, clothing, etc.) stored within the internal volume 325. In some embodiments, the oxidant source 330 can be disposed within a portion of the fabric 335 having an aperture 375 that permits ambient air external to the enclosure 300 to be drawn into the oxidant source 330. The oxidant source 330 can include one or more support structures 380 extending through the aperture 375 to prevent the fabric 335 from obstructing an air-intake port 385 of the oxidant source 330. Thus, the oxidant source 330 can be coupled to the sidewall 305A such that ambient air external to the enclosure 300 is drawn into the oxidant source 330 while oxidant discharged by the oxidant source 330 is simultaneously emitted from the oxidant source 330 and into the internal volume 325 to render microorganisms, like viruses, on the objects 370 (e.g., medical equipment) harmless, inert, or otherwise uncommunicable to persons and/or animals that come into contact with the objects. For example, the enclosure 300 may include a retaining structure (e.g., pocket) in one of the sidewalls. The retaining structure may include one or more walls to form a space sized and shaped to hold the oxidant source 330. One or more walls of the retaining structure may include mesh material to allow flow of gasses therethrough. For example, the wall(s) of the retaining structure facing one or more of the air intake port and air output port of the oxidant source 330 may be formed of a mesh material to allow free flow of gasses therethrough. A retaining structure may be disposed in any of the sidewalls 305a-305d, the ceiling 320, or the floor 315, such as a lower region of the sidewalls 305a-305d. As shown in FIGS. 3A and 3B, the retaining structure may be disposed in a lower region of the sidewall 305a. The retaining structure may be disposed in the internal volume or on the exterior of the enclosure 300 (where a portion of the sidewall is mesh to allow oxidant to flow into the internal volume 325).

FIG. 3C depicts one example of oxidant flow within the enclosure 300. In this example, the oxidant source 330 is disposed on the sidewall 305B and oriented to draw ambient air external to the enclosure 300 (as illustrated by flow line 390) into the oxidant source 330. The oxidant source 330 is also oriented such that a quantity of oxidant is propelled from an oxidant output port (e.g., oxidant output port 230 shown in FIGS. 2A and 2B) of the oxidant source 330 toward the ceiling 320 (as illustrated by flow line 395A). The portion of oxidant may settle downward, onto and/or around the objects 370 (e.g., medical equipment) within the internal volume 325 of the enclosure 300. The portion of oxidant propelled toward the ceiling 320 can drift along air currents (e.g., oxidant flow lines 395A, 395B) within the enclosure 300 created by one or more fans within the enclosure. For example, the oxidant source 330 can include one or more fans (e.g., fan 150).

Additionally, or alternatively, a distinct or independent fan can be disposed within the enclosure 300 to direct flow of oxidant within the internal volume 325. At least a portion of the oxidant propelled along flow lines 395A, 395B can come into contact with microorganism(s), scent source(s), or scent molecules within the enclosure 300, such as, microorganisms on the objects 370 or in the air within the enclosure 300. Thus, objects disposed within the internal volume 325 of the enclosure 300 can be treated to render microorganisms harmless to people subsequently handling or using the objects 370 and at least partially reduce the scent sources thereon. For example, a physician, nurse, phlebotomist, or other medical personnel can utilize the enclosure 300 to treat medical equipment previously exposed to microorganisms, such as viruses (e.g., COVID-19).

Exposing polymer, such as a transparent plastic, to an oxidant can degrade the polymer. For example the oxidant can cause a transparent polymer to become opaque. In some embodiments, oxidant can be propelled into the internal volume 325 in particular quantities and/or flow lines as to sufficiently treat microorganisms or scent sources on objects at least partially made of polymer (e.g., a transparent face-shield) without degrading or otherwise damaging the objects. For example, the fan can be positioned such that oxidant is propelled onto and past the objects but not permitted to collect on the surface of the objects for extended durations of time.

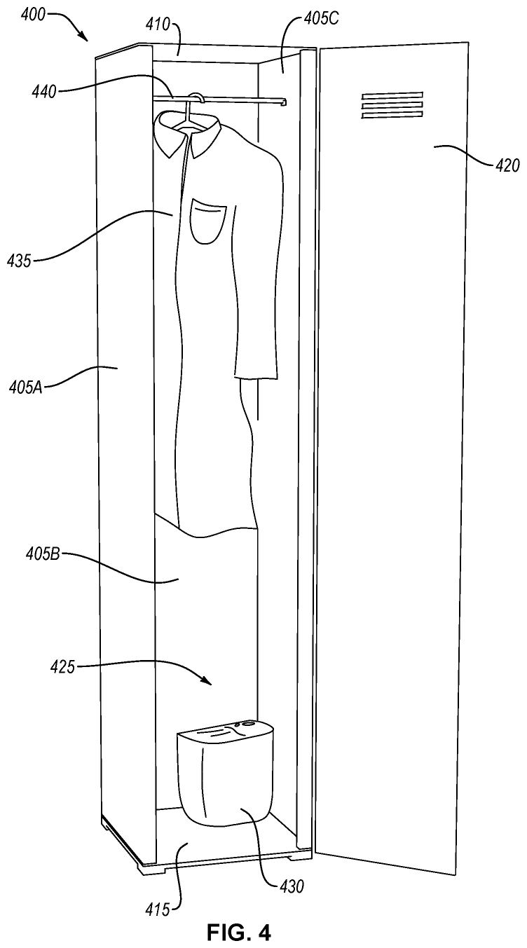

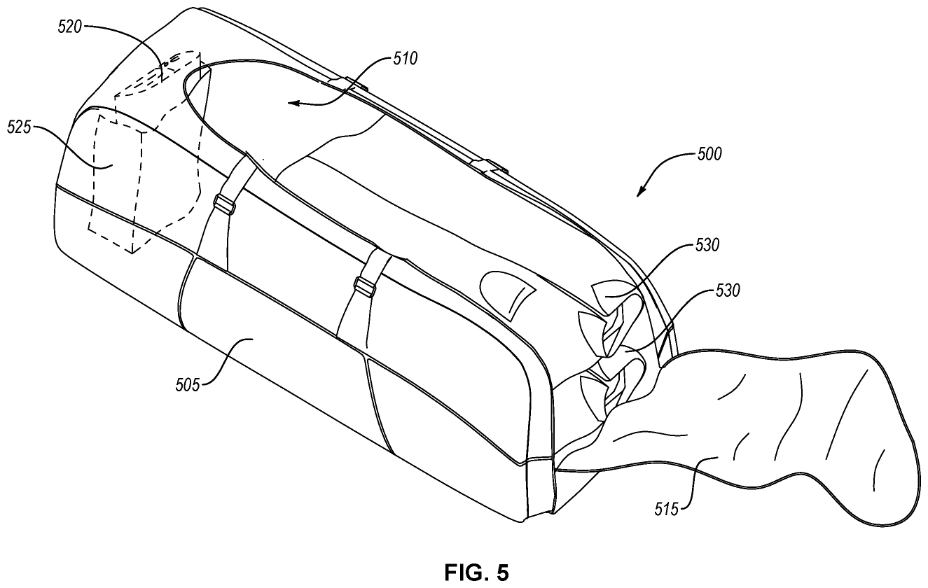

FIGS. 4-6 show various embodiments of enclosures for treating microorganisms, according to some embodiments. FIG. 4 illustrates an example enclosure 400, such as, a locker at a gym, school, hospital, or other building that requires personal lockers for its occupants. The enclosure 400 can include sidewalls 405A, 405B, 405C, a ceiling 410, a floor 415, and a door 420. The sidewalls 405A, 405B, 405C, ceiling 410, floor 415, and door 420 can form or define an internal volume 425 within which objects can be disposed. In some embodiments, a portable oxidant source 430 can be disposed within the internal volume 425 of the enclosure 400 to treat microorganisms within the internal volume 425. For example, an article of clothing 435 that has microorganisms or scent sources on a surface thereof can be placed within the enclosure 400 and subsequently treated to render the microorganisms harmless to a person who subsequently comes into contact with the article of clothing 435 or at least partially reduce (e.g., render inactive) scent sources thereon. The portable oxidant source 430 can be substantially similar to the oxidant source 105 and the portable oxidant source 200 described herein. For example, the portable oxidant source 430 can include the controller 140 operably coupled to at least one of the user interface module 145, the fan 150, the power supply 155, the communication module 160, or the oxidant generator 165.

In some embodiments, the article of clothing 435 can be hung from a bar 440 or other mechanism for retaining the article of clothing within the enclosure 400, such as, shelves, hooks, hangers, drawers, pegs, or combinations thereof. The article of clothing 435 can be a lab coat, scrubs, a jacket, a shirt, a hospital gown, or another article of clothing potentially exposed to microorganisms. Additionally, or alternatively, other objects can be disposed within the enclosure 400 and subsequently treated to render microorganisms thereon harmless. For example, medical equipment, blankets, sheets, safety glasses, power tools, hand tools, hard hats, electronic devices, or other objects can be disposed within the enclosure 400 and treated by the portable oxidant source 430 to render microorganisms on the objects harmless. Although not shown in FIG. 4, the enclosure 400 can, in some embodiments, include an aperture adjacent the portable oxidant source 430 such that air external to the enclosure 400 can be drawn into the portable oxidant source 430. For example, the aperture (not shown) can be formed within the sidewall 405B of the enclosure 400.

FIG. 5 shows another embodiment of an enclosure 500 for treating microorganisms, according to some embodiments. The enclosure 500 can be a gym bag, a piece of luggage, a backpack, or any other portable storage container. The enclosure 500 can include an outer surface 505 that forms or defines an internal volume 510. In some embodiments, the outer surface 505 can include a fabric, a polymer, a metal, or a combination thereof The internal volume 510 can be accessible via one or more releasable panels or flaps 515 of the outer surface 505. For example, the one or more releasable panels or flaps 515 can utilize zippers, hook and loop fastener, magnets, buttons, clasps, latches, or a combination thereof to releasably couple to the releasable panels or flaps 515 to the outer surface 510 of the enclosure 500.

In some embodiments, a portable oxidant source 520 can be disposed within the internal volume 510 of the enclosure 500 to treat microorganisms within the internal volume 510. For example, the portable oxidant source 520 can be disposed within a pocket or retaining structure 525 within the enclosure 500. The retaining structure 525 can be sewn or otherwise affixed to enclosure 500. In some embodiments, the enclosure 500 may not include the retaining structure 525. Instead, the portable oxidant source 520 can merely be disposed within the internal volume 510 of the enclosure 500. The portable oxidant source 520 can be utilized to treat objects (e.g., articles of clothing 530) within the enclosure 500 having microorganisms on a surface thereof to render the microorganisms harmless to a person who subsequently comes into contact with the objects. The portable oxidant source 520 can be substantially similar to the oxidant source 105 and the portable oxidant source 200 described herein. For example, the portable oxidant source 520 can include the controller 140 operably coupled to at least one of the user interface module 145, the fan 150, the power supply 155, the communication module 160, or the oxidant generator 165.

The articles of clothing 530 can be a lab coat, scrubs, a jacket, a shirt, a hospital gown, or another article of clothing potentially exposed to microorganisms. Additionally, or alternatively, other objects can be disposed within the enclosure 500 and subsequently treated to render microorganisms thereon harmless. For example, medical equipment, blankets, sheets, safety glasses, power tools, hand tools, hard hats, electronic devices, or other objects can be disposed within the enclosure 500 and treated by the portable oxidant source 520 to render microorganisms on the objects harmless. Although not shown in FIG. 5, the enclosure 500 can, in some embodiments, include an aperture or mesh section adjacent the portable oxidant source 520 such that air external to the enclosure 500 can be drawn into the portable oxidant source 520. For example, the outer surface 505 can include a mesh section that permits air to be drawn into the portable oxidant source 520 from adjacent the enclosure 500.

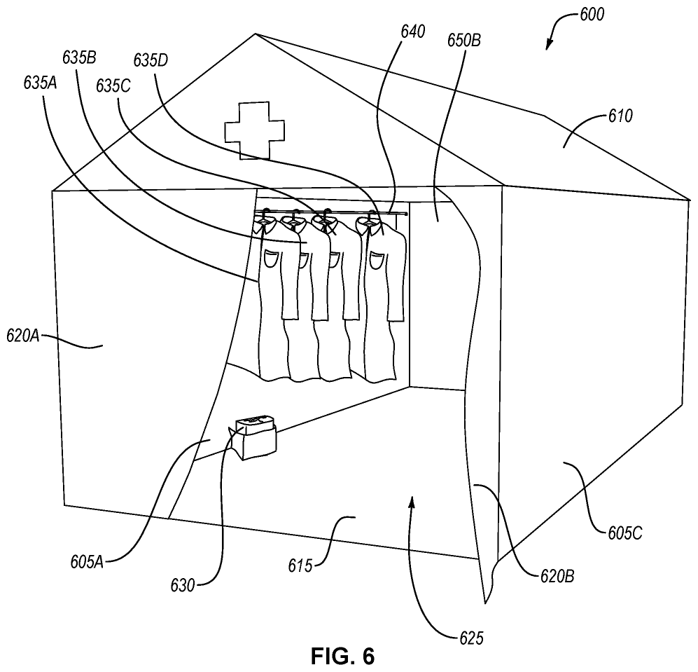

FIG. 6 illustrates an example enclosure 600, such as, a medical tent, military tent, temporary hospital, temporary housing, or other semi-permanent structure. The enclosure 600 can include sidewalls 605A, 605B, 605C, a roof 610, a floor 615, and one or more doors 620A, 620B. The sidewalls 605A, 605B, 605C, roof 610, floor 615, and door(s) 620A, 620B can form or define an internal volume 625 within which objects can be disposed. In some embodiments, a portable oxidant source 630 can be disposed within the internal volume 625 of the enclosure 600 to treat microorganisms within the internal volume 625. For example, the portable oxidant source 630 can be disposed within a pocket or retaining structure 645 within the enclosure 600. The retaining structure 645 can be sewn or otherwise affixed to enclosure 600. In some embodiments, the enclosure 600 may not include the retaining structure 645. Instead, the portable oxidant source 630 can merely be disposed within the internal volume 625 of the enclosure 600. One or more of the articles of clothing 635A-D can include microorganisms thereon and can be placed within the enclosure 600 to treat or otherwise render the microorganisms harmless to a person who subsequently comes into contact with the articles of clothing 635A-D. The portable oxidant source 630 can be substantially similar to the oxidant source 105 and the portable oxidant source 200 described herein. For example, the portable oxidant source 630 can include the controller 140 operably coupled to at least one of the user interface module 145, the fan 150, the power supply 155, the communication module 160, or the oxidant generator 165.

In some embodiments, the articles of clothing 635A-D can be hung from a bar 640 or other mechanism for retaining the articles of clothing 635A-D within the enclosure 600, such as, shelves, hooks, hangers, drawers, pegs, or combinations thereof The articles of clothing 635A-D can be lab coats, scrubs, a jacket, a shirt, a hospital gown, or another article of clothing potentially exposed to microorganisms. Additionally, or alternatively, other objects can be disposed within the enclosure 600 and subsequently treated to render microorganisms thereon harmless. For example, medical equipment, blankets, sheets, safety glasses, power tools, hand tools, hard hats, electronic devices, or other objects can be disposed within the enclosure 600 and treated by the portable oxidant source 430 to render microorganisms on the objects harmless. Although not shown in FIG. 6, the enclosure 600 can, in some embodiments, include an aperture adjacent the portable oxidant source 630 such that air external to the enclosure 600 can be drawn into the portable oxidant source 630. For example, the aperture (not shown) can be formed within the sidewall 605A of the enclosure 600.

Any of the enclosures disclosed herein (e.g., enclosures 300, 400, 500, and 600) can include an oxidant source which generates and propels oxidant throughout the internal volume of the enclosure. Additionally, or alternatively, the oxidant source can operate in a fan-only mode which draws ambient air (e.g., air external to the enclosure) into the enclosure and circulates the ambient air to reduce residual oxidant within the enclosure and within the oxidant source. In the fan-only mode, the oxidant source may not generate oxidant and therefore only propel the ambient air throughout the enclosure. In some examples, the oxidant source may produce a very limited quantity of oxidant during the fan-only mode to treat the air within the enclosure but no longer treat objects within the enclosure. Reducing the amount of residual oxidant within the enclosure can be beneficial. For example, reducing the amount of residual oxidant within the enclosure can prevent damage to the enclosure and/or objects stored therein by preventing prolonged exposure to a relatively high concentration of oxidant.

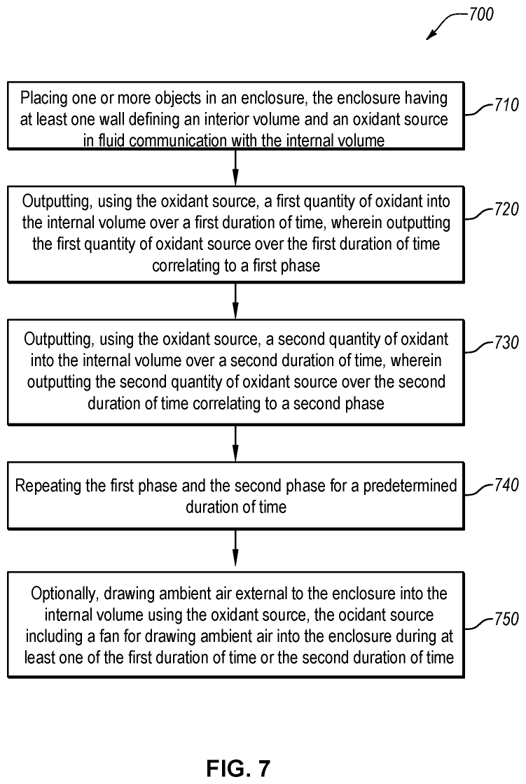

FIG. 7 is a flow diagram of a method 700 of treating objects within an enclosure, according to at least some embodiments. The method 700 includes the act 710 of placing objects in an enclosure, the enclosure having at least one wall defining an interior volume and an oxidant source in fluid communication with the internal volume. The method 700 includes the act 720 of outputting, using the oxidant source, a first quantity of oxidant into the internal volume over a first duration of time, the first quantity of oxidant source over the first duration of time correlating to a first phase. The method 700 includes the act 730 of outputting, using the oxidant source, a second quantity of oxidant into the internal volume over a second duration of time, the second quantity of oxidant source over the second duration of time correlating to a second phase. The method 700 includes the act 740 of repeating the first phase and the second phase for a predetermined duration of time. Optionally, the method 700 includes the act 750 of drawing ambient air external to the enclosure into the internal volume using the oxidant source, the oxidant source including a fan for drawing ambient air into the enclosure during at least one of the first duration of time or the second duration of time. Accordingly, the method 700 may treat microorganisms within an enclosure. The method 700 may include more or fewer acts than the acts 710-750. For example, the method 700 may not include the act 710 or 750.

The method 700 includes the act 710 of placing objects in an enclosure, the enclosure having at least one wall defining an interior volume and an oxidant source in fluid communication with the internal volume. The enclosure can be any of the enclosures (e.g., enclosure 100, 300, 400, 500, 600) disclosed herein. In examples, the enclosure may include any components of any of the enclosures disclosed herein. For example, the enclosure may include one or more shelves, hooks, hangers, drawers, pegs, or any other mechanism for retaining equipment/objects within the enclosure, as disclosed herein. The objects placed in the enclosure (e.g., the internal volume) can be anything that can fit within the enclosure, such as, clothing, personal protective equipment, tools, medical equipment, masks, aprons, face shields, smocks, gloves, hats, hunting equipment, and so on. As previously described herein, the enclosure can define or form the internal volume, for example, between one or more sidewalls, doors, ceilings, and floors of the enclosure.

The oxidant source can be positioned in fluid communication with the internal volume, for example, by disposing the oxidant source within the internal volume and/or disposing an oxidant output port of the oxidant source within the internal volume. In some embodiments, the oxidant source can be disposed within a retaining structure (e.g., retaining structures 525, 645) affixed to a sidewall or other portion of the enclosure. The oxidant source can be the portable oxidant source 200 (FIGS. 2A-C), or any other oxidant source disclosed herein (e.g., oxidant source 105). In examples, the oxidant source may include any components of any of the oxidant sources disclosed herein. For example, the oxidant source may include the user input region (e.g., user input region 220) as disclosed herein. The user input region may include one or more inputs, such as buttons, switches, knobs, indicators, or toggles, for activating the oxidant source, deactivating the oxidant source, selecting a mode of operation, increasing or decreasing an output of the oxidant source, viewing an attribute of the oxidant source, or directing any other operation of the oxidant source.

The method 700 includes the act 720 of outputting, using the oxidant source, a first quantity of oxidant into the internal volume over a first duration of time, the first quantity of oxidant source over the first duration of time correlating to a first phase. In some embodiments, the oxidant can be output or generated from an oxidant output port (e.g., oxidant output port 230 shown in FIGS. 2A and 2B) of the oxidant source. For example, the oxidant source can include an ozone generator, such as, a corona discharge ozone generator (e.g., corona discharge plate), an ultraviolet ozone generator, an electrolytic ozone generator, or any other type of ozone generator. In some examples, the oxidant source can include an ionizer or electrostatic precipitator.

As such, outputting the first quantity of oxidant into the internal volume can include outputting one or more of ozone, diatomic oxygen, diatomic halogens, peroxides, radicals of any of the foregoing or components thereof, metastable oxygen, negatively charged metal oxides, encapsulated ozone, activated ozone, peracetic acid, chlorine dioxide, thixotropic gels, singlet oxygen, hypochlorite, or chlorite, from the oxidant source. For example, outputting a first quantity of oxidant may include outputting ozone from a portable ozone generator at a selected oxidant (ozone) output rate. Even more specifically, outputting the oxidant from the oxidant source at the selected oxidant output rate may include outputting ozone from the corona discharge ozone generator of the oxidant source.

In some embodiments, the oxidant source can operate based on an operational program that causes the oxidant source to output the first quantity of oxidant over the first duration of time. As a non-limiting example, the operational program may cause the oxidant source to output 60 mg of oxidant (e.g., the first quantity of oxidant) per cubic meter over a 3.5 minute duration of time (e.g., the first duration of time), which is an oxidant output rate of approximately 1030 mg per cubic meter per hour. Outputting the first quantity of oxidant into the internal volume over a first duration of time may include outputting the first quantity of oxidant at a selected output rate, such as any of the output rates disclosed herein. The first output rate may be a highest output rate.

In some embodiments, the first duration of time can range from 5 minutes, such as 5 minutes to 4 hours, 10 minutes to 2 hours, 15 minutes to 1 hour, 30 minutes to 1.5 hours, 1 hour to 3 hours, less than 4 hours, more than 1 hour, or more than 2 hours. Outputting the first quantity of oxidant for the first duration of time can be considered or correlate with the first phase.

The method 700 includes the act 730 of outputting, using the oxidant source, a second quantity of oxidant into the internal volume over a second duration of time, the second quantity of oxidant source over the second duration of time correlating to a second phase. In some embodiments, outputting the second quantity of oxidant into the internal volume can include outputting one or more of ozone, diatomic oxygen, diatomic halogens, peroxides, radicals of any of the foregoing or components thereof, metastable oxygen, negatively charged metal oxides, encapsulated ozone, activated ozone, peracetic acid, chlorine dioxide, thixotropic gels, singlet oxygen, hypochlorite, or chlorite, from the oxidant source. For example, outputting the second quantity of oxidant may include outputting ozone from a portable ozone generator at a selected oxidant (ozone) output rate. Even more specifically, outputting the oxidant from the oxidant source at the selected oxidant output rate may include outputting ozone from the corona discharge ozone generator of the oxidant source.

In some embodiments, the oxidant source can operate based on the operational program that also causes the oxidant source to output the second quantity of oxidant over the second duration of time. As a non-limiting example, the operational program may cause the oxidant source to output 4 mg of oxidant (e.g., the second quantity of oxidant) per cubic meter over a 1 minute duration of time (e.g., the second duration of time), which is an oxidant output rate of approximately 240 mg per cubic meter per hour. Outputting the second quantity of oxidant into the internal volume over a second duration of time may include outputting the second quantity of oxidant at a selected output rate, such as any of the output rates disclosed herein. The second output rate may be lower than the first output rate.

In some embodiments, the second duration of time can range from 1 minutes, such as 1 minutes to 2 hours, 2 minutes to 1 hours, 5 minutes to 30 minutes, 10 minutes to 20 minutes, less than 2 hours, more than 30 minutes, or more than 1 hour. Outputting the second quantity of oxidant for the second duration of time can be considered or correlate with the second phase.

The method 700 includes the act 740 of repeating the first phase and the second phase for a predetermined duration of time. Accordingly, the operational program may cause the oxidant source to output oxidant at a relatively high rate for the first duration of time (i.e., the first phase), subsequently output oxidant at a relatively low rate for the second duration of time (i.e., the second phase), and cyclically repeat the first and second phases for the pre-determined duration of time.

For example, the oxidant source can output 60 mg of oxidant per cubic meter over a 3.5 minute duration of time during the first phase (e.g., a relatively high oxidant output rate of approximately 1030 mg per cubic meter per hour), subsequently output 4 mg of oxidant per cubic meter over a 1 minute duration of time during the second phase (e.g., a relatively low oxidant output rate of approximately 240 mg per cubic meter per hour), and then repeat each of the first and second phases 5 times such that the predetermined duration of time is about 22.5 minutes. By alternating between the first and second phases, an item within the enclosure can be protected from damage caused by constant or near constant exposure to high concentrations of oxidant while still treating microorganisms on the item.

In examples, the method 700 optionally includes the act 750 of drawing ambient air external to the enclosure into the internal volume using the oxidant source, the oxidant source drawing ambient air into the enclosure during at least one of the first duration of time or the second duration of time. The fan can be one or more fans, such as an intake fan, a cooling fan, an output fan, or combination thereof. Exemplary fans include microfans, centrifugal fans, cyclonic blowers, etc. Each fan may be operably coupled to a power supply and a controller, to activate, adjust speed, and deactivate according to operational instructions. In examples, air that is drawn into the oxidant source can be mixed with an oxidant generated by an oxidant generator and propelled from the oxidant source into the internal volume of the enclosure.

In examples, the method 700 can include or otherwise incorporate multiple oxidant sources. For example, the method 700 can include two distinct oxidant sources, each in fluid communication with the internal volume of the enclosure. Each of the multiple oxidant sources can operate under the same or distinct operational programs to implement the method 700. Additionally, or alternatively, the multiple oxidant sources can be communicatively coupled via communication modules within each of the multiple oxidant sources.

In examples, the method 700 may include operating the oxidant source such that the fan is operating while the oxidant generator is not generating oxidant. For example, ozone may remain on the corona discharge coils of a corona discharge ozone generator. In such examples, the ozone may degrade the coils if left in place. Ozone degradation may cause the ozone generator to lose efficiency and drain the battery of the oxidant source. A short purge with ambient air may help void the coils of any ozone after production of ozone is halted. The fan may remain in operation for at least at least 1 second after the portable oxidant source has ceased producing oxidant, such as 2 seconds to 2 minutes, 3 seconds to 10 seconds, 5 seconds to 15 seconds, 10 seconds to 20 seconds, 15 seconds to 30 seconds, 2 seconds to 30 seconds, 30 seconds to 1 minute, 1.5 minutes, 1.5 minutes to 2 minutes, less than 2 minutes, or less than 1 minute after the oxidant source has ceased producing oxidant. Thus, the fan can remain in operation at the conclusion of the method 700 to void residual oxidant from settling or collecting within the oxidant source.

In some examples, the method 700 may include outputting, using the oxidant source, a third quantity of oxidant into the internal volume over a third duration of time may include outputting the third quantity of oxidant at a selected output rate, such as any of the output rates disclosed herein. The third output rate may be lower than the first output rate but higher than the second. The third quantity may be lower than the first quantity and higher than the second quantity. The third duration may include any of the durations disclosed herein.

Any one or more of the acts 710-750 of the method 700 can be initiated and/or regulated by a controller (e.g., the controller 140) disposed within the oxidant source. For example, outputting a first quantity of oxidant into the internal volume over a first duration of time, outputting a second quantity of oxidant into the internal volume over a second duration of time, and repeating the first phase and the second phase for a predetermined duration of time can include controlling the output of the oxidant source using a controller operably coupled to the oxidant source.

The methods, devices, and systems disclosed herein can be used to treat objects to reduce, remove, or render contaminants thereon inert. For example, the method 700, devices, and systems disclosed herein can be used to treat microorganisms, scent sources, or scent material on objects within the enclosure while providing a reduced oxidant concentration for durations to prevent damage to the objects while also maintaining at least a minimum amount of oxidant input to prevent untreated input of ambient air into the internal volume of the enclosure. Thus, the objects can be effectively treated with oxidants such as ozone to eliminate contaminants (e.g., kill microorganisms or eliminate scent sources) thereon without damaging the objects, all while preventing reintroduction of contaminants onto the objects during.

In some examples, the endpoint values disclosed herein may be approximate values, which may vary by 10% or less from the precise endpoint value given. In such examples, the term “about” or “substantially” may indicate the approximate values.

Aspects of any of the examples disclosed herein may be used with aspects of any other examples, disclosed herein without limitation.

While various aspects and embodiments have been disclosed herein, other aspects and embodiments are contemplated. The various aspects and embodiments disclosed herein are for purposes of illustration and are not intended to be limiting. Additionally, the words “including,” “having,” and variants thereof (e.g., “includes” and “has”) as used herein, including the claims, shall be open ended and have the same meaning as the word “comprising” and variants thereof (e.g., “comprise” and “comprises”).

What is claimed is:

1. An apparatus for treating objects, the apparatus comprising:
an enclosure having at least one wall defining an internal volume;
an oxidant source operably coupled to the at least one wall of the enclosure and configured to output an oxidant within the internal volume; and
a controller operably coupled to the oxidant source, the controller being configured to cause the oxidant source to cyclically discharge the oxidant within the internal volume.

2. The apparatus of claim 1, wherein the controller is configured to cause the oxidant source to discharge a first quantity of oxidant into the internal volume over a first duration of time and a second quantity of oxidant into the internal volume over a second duration of time to cyclically discharge the oxidant.

3. The apparatus of claim 2, wherein the controller is configured to cause the oxidant source to discharge a third quantity of oxidant into the internal volume over a third duration of time and a fourth quantity of oxidant into the internal volume over a fourth duration of time to cyclically discharge the oxidant.

4. The apparatus of claim 2, wherein:
the first quantity of oxidant is greater than the second quantity of oxidant; and
the first duration of time is greater than the second duration of time.

5. The apparatus of claim 3, wherein:
the first quantity of oxidant and the third quantity of oxidant are equivalent;
the second quantity of oxidant and the fourth quantity of oxidant are equivalent;
the first duration of time and the third duration of time are equivalent; and
the second duration of time and the fourth duration of time are equivalent.

6. An enclosure for treating objects, comprising:
at least one wall defining an internal volume;
an oxidant source operably coupled to the at least one wall and configured to output an oxidant within the internal volume; and
a controller operably coupled to the oxidant source, the controller comprising:
a computer-readable medium having executable instructions; and
a processor coupled to the computer-readable medium and configured to execute the executable instructions, wherein when executed, the executable instructions cause the controller to perform the operations of:
outputting, using the oxidant source, a first quantity of oxidant into the internal volume over a first duration of time, the first quantity of oxidant source over the first duration of time correlating to a first phase; and
outputting, using the oxidant source, a second quantity of oxidant into the internal volume over a second duration of time, the second quantity of oxidant source over the second duration of time correlating to a second phase; and
repeating the first phase and the second phase for a predetermined duration of time to treat microorganisms within the internal volume.

7. The enclosure of claim 6, wherein the oxidant source includes a corona discharge ozone generator.

8. The enclosure of claim 6, wherein the oxidant source includes a source of one or more of ozone, diatomic oxygen, diatomic halogens, peroxides, radicals of any of the foregoing or components thereof, metastable oxygen, negatively charged metal oxides, encapsulated ozone, activated ozone, peracetic acid, chlorine dioxide, thixotropic gels, singlet oxygen, hypochlorite, or chlorite.

9. The enclosure of claim 6, wherein the first quantity of oxidant is at least about 60 mg and the first duration of time is about 3 minutes.

10. The enclosure of claim 6, wherein the second quantity of oxidant is about 6 mg or less and the second duration of time is about 1 minute.

11. The enclosure of claim 6, wherein:
the first quantity of oxidant is greater than the second quantity of oxidant; and
the second first duration of time is greater than the second duration of time.

12. The enclosure of claim 6, wherein the oxidant source is configured to draw ambient air external to the enclosure into the internal volume during at least one of the first duration of time or the second duration of time.

13. The enclosure of claim 6, wherein the predetermined duration of time is about 30 minutes.

14. The enclosure of claim 6, wherein the oxidant source includes a fan positioned to propel oxidant away from the oxidant source.

15. The enclosure of claim 6, further comprising:
a power supply operably coupled to the oxidant source and the controller; and
a housing containing the power supply, the oxidant source, and the controller.

16. A method of treating objects within an enclosure, the method comprising:
placing one or more objects in an enclosure, the enclosure having at least one wall defining an interior volume and an oxidant source in fluid communication with the internal volume, and
outputting, using the oxidant source, a first quantity of oxidant into the internal volume over a first duration of time, the first quantity of oxidant source over the first duration of time correlating to a first phase;
outputting, using the oxidant source, a second quantity of oxidant into the internal volume over a second duration of time, the second quantity of oxidant source over the second duration of time correlating to a second phase; and
repeating the first phase and the second phase for a predetermined duration of time.

17. The method of claim 16, wherein the oxidant source is operably coupled to the at least one wall.

18. The method of claim 17, wherein the oxidant source includes a corona discharge ozone generator and the oxidant includes ozone.

19. The method of claim 16, wherein the first quantity of oxidant is at least about 60 mg and the first duration of time is about 3 minutes.

20. The method of claim 16, wherein the second quantity of oxidant is about 6 mg or less and the second duration of time is about 1 minute.

21. The method of claim 16, wherein:
the first quantity of oxidant is greater than the second quantity of oxidant; and
the first duration of time is greater than the second duration of time.

22. The method of claim 16, further comprising drawing ambient air external to the enclosure into the internal volume using the oxidant source, the oxidant source including a fan for drawing ambient air into the enclosure during at least one of the first duration of time or the second duration of time.

23. The method of claim 16, wherein the predetermined duration of time is about 30 minutes.

24. The method of claim 16, wherein the oxidant includes one or more of ozone, diatomic oxygen, diatomic halogens, peroxides, radicals of any of the foregoing or components thereof, metastable oxygen, negatively charged metal oxides, encapsulated ozone, activated ozone, peracetic acid, chlorine dioxide, thixotropic gels, singlet oxygen, hypochlorite, or chlorite.

25. The method of claim 16, wherein placing one or more objects into the interior volume includes placing one or more of clothing, face masks, eyeglasses, face shields, aprons, smocks, gloves, or hats into the internal volume.

26. The method of claim 16, wherein outputting a first quantity of oxidant into the internal volume over a first duration of time, outputting a second quantity of oxidant into the internal volume over a second duration of time, and repeating the first phase and the second phase for a predetermined duration of time includes controlling the output of the oxidant source using a controller operably coupled to the oxidant source.
